(19) United States
(12) Patent Application Publication
(10) Pub. No.: US 2025/0319227 A1
(43) Pub. Date: Oct. 16, 2025

(54) Aroma diffuser

(71) Applicant: AROMA PERFECTO LLC, Diamond Bar, CA (US)

(72) Inventor: Yong WU, Guangzhou (CN)

(21) Appl. No.: 19/175,392

(22) Filed: Apr. 10, 2025

(30) Foreign Application Priority Data
Apr. 10, 2024 (CN) 202420728844.7

(51) Int. Cl. A61L 9/14

(52) U.S. Cl. CPC A61L 9/14; A61L 2209/134

(57) ABSTRACT

An aroma diffuser includes a limiting device provided between an atomizing assembly and a barrel body, with at least one sealing member provided on the limiting device. When the atomizing assembly is connected to the barrel body, the sealing member is configured for horizontally sealing a gap between the barrel body and the atomizing assembly. In this way, when the atomizing assembly and the barrel body are installed or disassembled in a rotating manner, the sealing member will not be distorted and deformed along with the rotation, so that a sealing effect between the atomizing assembly and an inner barrel will not be affected, thereby effectively ensuring the airtightness of an airflow channel located inside the inner barrel.

### CROSS REFERENCE TO RELATED APPLICATIONS

The present application claims priority from Chinese Application No. CN 202420728844.7 filed on Apr. 10, 2024, all of which are hereby incorporated herein by reference.

### TECHNICAL FIELD

The present disclosure relates to the technical field of aroma diffusers, in particular to an aroma diffuser.

### BACKGROUND

An aroma diffuser, also known as an aromatherapy machine or air purifier, is equipment capable of converting liquid essential oils into gas molecules that diffuse and volatilize into air.

To make full use of a space to place an air pump, the existing aroma diffuser adopts an arrangement where an atomizing assembly is located at the upper part and an air pump is located at the lower part. A main structure includes a barrel body provided with an inner barrel and an outer barrel, and an atomizing assembly connected to the top of the barrel body. An atomizing core is provided in the atomizing assembly. An essential oil bottle is contained in the inner barrel. The air pump is contained in an outer barrel space at the bottom of the inner barrel. An outlet of the air pump communicates with a cavity of the inner barrel through a gas guide pipe. A liquid channel connected to the essential oil bottle and a gas channel communicating with the cavity of the inner barrel are respectively arranged on the atomizing core. An airflow generated by the air pump is sprayed out of a gas outlet of the atomizing core, forming a negative pressure to suck essential oil in the essential oil bottle into a liquid outlet of the atomizing core. After being mixed with a gas and atomized, the essential oil is discharged from a fog outlet of the atomizing assembly. Since the air is introduced through the cavity of the inner barrel, a sealing effect between the barrel body and the atomizing assembly determines the amount of the air entering the atomizing core. Moreover, to replace the essential oil bottle, the barrel body and the atomizing assembly are mostly connected by threads or snapped connection, which will affect the sealing effect by the operations during disassembly and installation.

Therefore, the Chinese patent document for utility model with publication No. CN220142248U discloses a novel aroma diffuser structure. The novel aroma diffuser structure includes a base and an outer body, wherein the outer body internally includes an upper cavity and a lower cavity, an atomizing core frame is connected to an upper side of the upper cavity, and an inner barrel sealing ring is annularly fixed at an upper end of the atomizing core frame. The inner barrel sealing ring functions to improve the airtightness between the atomizing core frame and the inner barrel, and prevent air in the cavity of the inner barrel from escaping through a gap between the atomizing core frame and the inner barrel and affecting the gas supply to the atomizing core. An angular boss is also provided at an inner side of the top of the inner barrel. When the atomizing core frame is fixed on the inner barrel, the angular boss presses the inner barrel sealing ring to ensure the airtightness between the atomizing core frame and the inner barrel. However, in this scheme, the inner barrel sealing ring is installed on a side wall of the atomizing core frame, and the sealing effect can only be achieved through oblique extrusion between the angular boss and the inner barrel sealing ring. Since the atomizing core frame is in threaded connection with the inner barrel, when the atomizing core frame and the inner barrel are connected or disassembled in a rotating manner, the angular boss will inevitably drive the inner barrel sealing ring to twist and hence deform along the rotation due to oblique extrusion between the angular boss and the inner barrel sealing ring, and may even cause the inner barrel sealing ring to fall off from the side wall of the atomizing core frame, thereby affecting the sealing effect between the atomizing core frame and the inner barrel. To solve the problem of air leakage, a plurality of sealing rings are further used in the prior art to seal a barrel body and an atomizing assembly. Such a scheme also has the problems of complicated installation, and untight sealing due to the shifting displacement of the sealing ring.

The technical problem to be solved in the present application is to design an aroma diffuser that will not cause a sealing member to be distorted and deformed when an atomizing device is installed or disassembled, and can effectively ensure a sealing effect between the atomizing device and an inner barrel.

### SUMMARY

In order to overcome the defects of the prior art, an object of the present disclosure is to provide an aroma diffuser that will not cause a sealing member to be distorted and deformed when an atomizing device is installed or disassembled, and can effectively ensure a sealing effect between the atomizing device and an inner barrel.

A technical solution adopted by the present disclosure is as follows: an aroma diffuser includes an atomizing assembly and a barrel body that are connected to each other, wherein the barrel body includes an outer barrel and an inner barrel provided in the outer barrel, an airflow channel for an airflow to pass through is provided in the inner barrel, a limiting device is arranged between the atomizing assembly and the barrel body, at least one sealing member is provided on the limiting device, and the sealing member is configured for horizontally sealing a gap between the barrel body and the atomizing assembly when the atomizing assembly is connected to the barrel body. The sealing member may be a sealing ring, a sealing loop or other similar structures, and may have a circular, oval or quadrilateral cross section, etc.

In some embodiments, the limiting device is provided on a top surface of the outer barrel, and the sealing member is configured for horizontally sealing a gap between the top surface of the outer barrel and the atomizing assembly.

In some embodiments, the limiting device is provided on a top surface of the inner barrel, and the sealing member is configured for horizontally sealing a gap between the top surface of the inner barrel and the atomizing assembly.

In some embodiments, the atomizing assembly includes an end cover and a base, an atomizing core is provided in the base, the airflow channel is communicated with the atomizing core, the limiting device is provided on the end cover, and the sealing member is configured for horizontally sealing a gap between the barrel body and the end cover.

In some embodiments, the atomizing assembly includes an end cover and a base, an atomizing core is provided in the base, the airflow channel is communicated with the atomizing core, the limiting device is provided on the base, and the sealing member is configured for horizontally sealing a gap between the barrel body and the base.

In some embodiments, the limiting device is an annular groove, and the sealing member is provided in the annular groove and has a height greater than the depth of the annular groove.

In some embodiments, the limiting device is an annular boss, and a groove that matches with the annular boss is formed in the bottom of the sealing member.

In some embodiments, the atomizing assembly includes an end cover and a base, a limiting raised ring is provided at a lower end of the end cover; and the limiting raised ring is limited between the inner barrel and the base when the atomizing assembly is connected to the barrel body.

In some embodiments, a positioning member is provided between an inner wall of the outer barrel and an outer wall of the inner barrel.

Specifically, the positioning member includes a positioning groove formed in the inner wall of the outer barrel/the outer wall of the inner barrel, and a positioning block provided on the outer wall of the inner barrel/the inner wall of the outer barrel, and the positioning block is in sliding fitting with the positioning groove.

In some embodiments, a clamping member is provided between the inner wall of the outer barrel and the outer wall of the inner barrel.

Specifically, the positioning member includes a clamping groove formed in the inner wall of the outer barrel/the outer wall of the inner barrel, and a clamping lug provided on the outer wall of the inner barrel/the inner wall of the outer barrel, and the clamping lug is in snap fitting with the clamping groove.

The positioning member and the clamping member are provided between the outer barrel and the inner barrel, enabling the two positioning to be located relative to each other and matched closely, and thus, the inner barrel not rotate or fall out relative to the outer barrel.

In some embodiments, a hanging structure which is over-turned outward is provided at a rim of the inner barrel, and the hanging structure is configured for hanging at a rim of the outer barrel.

In this way, the inner barrel may be hung on the outer barrel and installed in conjunction with the positioning member and the clamping member. Preferably, the hanging structure, the positioning groove and the clamping lug are integrally formed with the inner barrel, and the single integrated structure of the inner barrel can achieve the functions such as hanging, positioning and clamping at the same time.

In some embodiments, an air inlet pipe that is communicated with the airflow channel is also provided at the bottom of the inner barrel, and an air outlet end of the air inlet pipe is higher than a bottom surface of the inner barrel, which can prevent a liquid leaking into the inner barrel from entering the air inlet pipe. Preferably, a baffle is provided above the air outlet end. Preferably, the air inlet pipe and the baffle may also be integrally formed with the inner barrel.

To facilitate installation, friction surfaces are distributed on an outer surface of the atomizing assembly, and the friction surfaces may be vertical stripes or diamond patterns or similar structures distributed on the end cover.

The present disclosure has the following beneficial effects: the limiting device is provided between the barrel body and the atomizing assembly, and at least one sealing member is provided in the limiting device and configured for horizontally sealing the gap between the barrel body and the atomizing assembly. When the atomizing assembly is connected to the barrel body, the inner barrel and the atomizing assembly are horizontally fitted with the upper and lower ends of the sealing member, respectively. In this way, when the atomizing assembly and the barrel body are installed or disassembled in a rotating manner, the sealing member will not be distorted and deformed along the rotation, so that a sealing effect between the atomizing assembly and the inner barrel will not be affected, thereby effectively ensuring the airtightness of the airflow channel inside the inner barrel.

### BRIEF DESCRIPTION OF THE DRAWINGS

FIG. 1 is a schematic structural view of an aroma diffuser according to an embodiment of the present disclosure;

FIG. 2 is a schematic view of an internal structure of the aroma diffuser;

FIG. 3 is a schematic view of a disassembled structure at a junction between an end cover and an inner barrel of the aroma diffuser;

FIG. 4 is a schematic sectional view of the aroma diffuser;

FIG. 5 is a large view of Part A in FIG. 4;

FIG. 6 is a schematic view of a disassembled structure of an atomizing assembly of the aroma diffuser;

FIG. 7 is a schematic view of a bottom structure of a fog baffle frame of the aroma diffuser;

FIG. 8 is a schematic structural view of an end cover of the aroma diffuser;

FIG. 9 is a schematic structural view of an atomizing core of the aroma diffuser;

FIG. 10 is an installation view of a sealing member of an aroma diffuser according to an embodiment of the present disclosure;

FIG. 11 is an installation view of a sealing member of an aroma diffuser according to another embodiment of the present disclosure; and

FIG. 12 is an installation view of a base of an aroma diffuser according to an embodiment of the present disclosure.

Reference signs: 1—atomizing assembly; 2—barrel body; 3—sealing member; 10—end cover; 11—base; 20—outer barrel; 21—inner barrel; 22—oil storage bottle; 12—atomizing core; 121—air outlet; 122—liquid outlet; 13—oil suction pipe; 4—airflow channel; 110—air inlet hole; 23—air pump; 230—air conveying pipe; 101—limiting raised ring; 210—annular limiting groove; 102—annular table; 112—partition plate; 113—diffusion hole; 114—gasket; 14—fog baffle frame; 140—fog baffle plate; 141—fog outlet; 142—limiting column; 115—limiting hole; 16—fog outlet channel; 17—fog outlet hole; 5—battery assembly; 6—base; 231—air outlet pipe; 7—limiting groove; 8—annular raised line; 30—annular groove; 116—upper connecting portion; and 117—lower connecting portion.

### DETAILED DESCRIPTION

The technical solutions in the embodiments of the present disclosure will be described clearly and completely in conjunction with the accompanying drawings in the embodiments of the present disclosure. Apparently, the described embodiments are merely some embodiments, rather than all embodiments, of the present disclosure. Based on the embodiments of the present disclosure, all other embodiments derived by an ordinary skill in the art without creative efforts shall fall within the protection scope of the present disclosure.

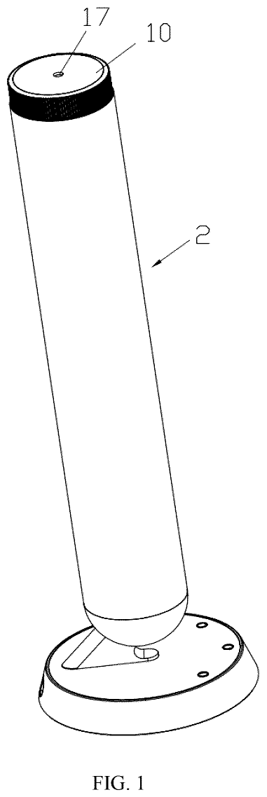

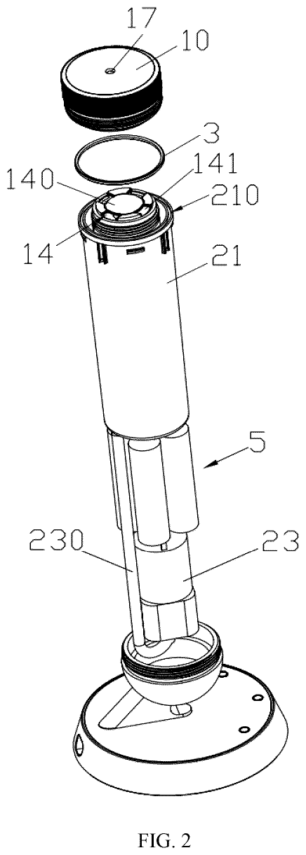

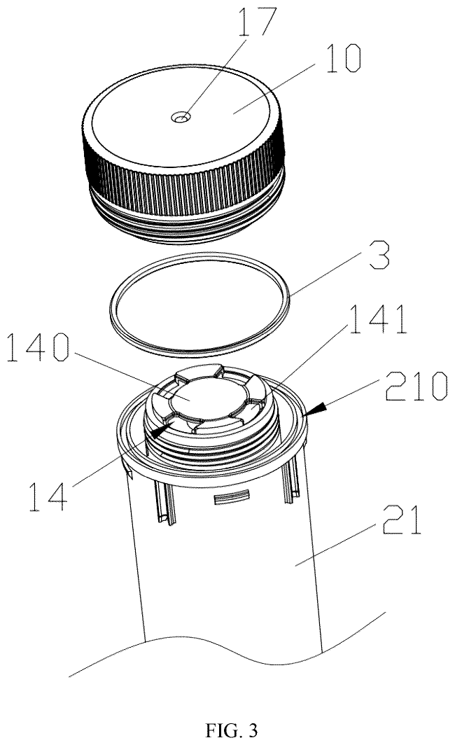

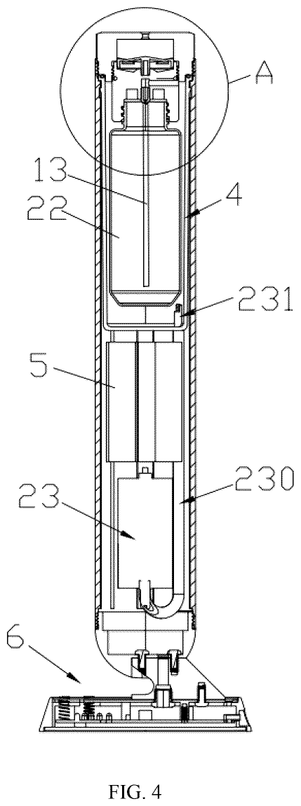

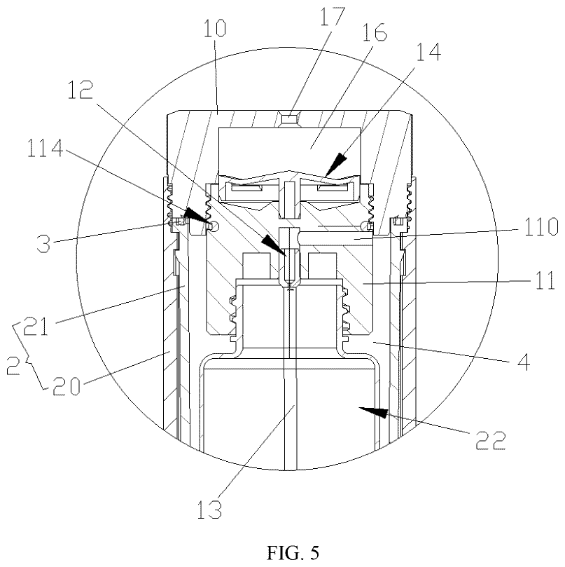

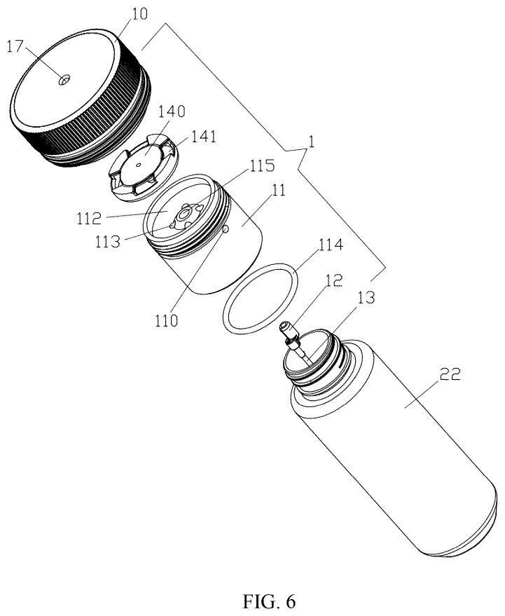

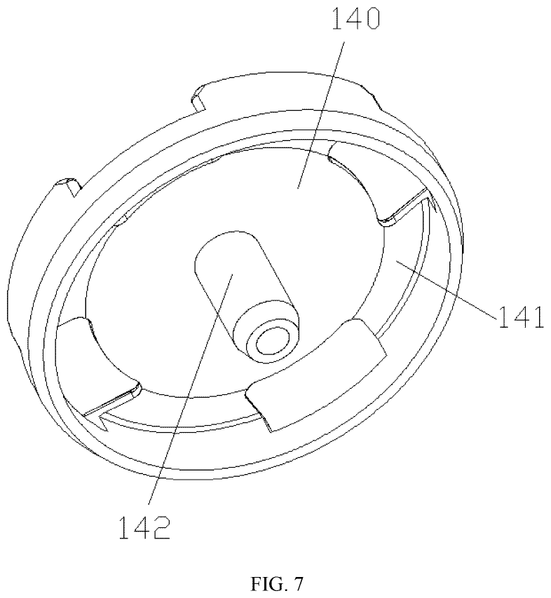

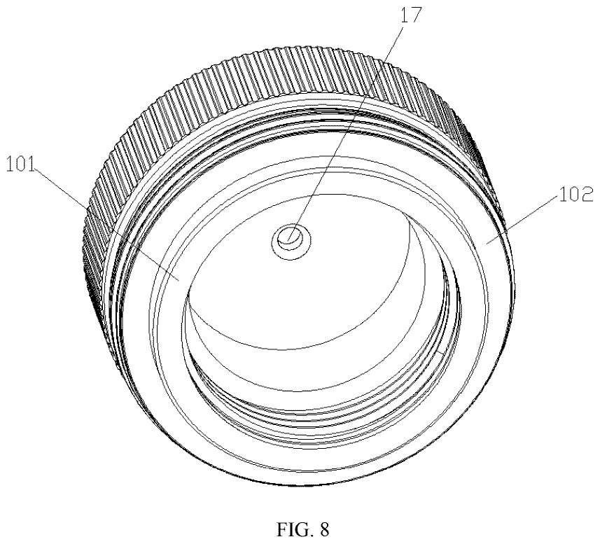

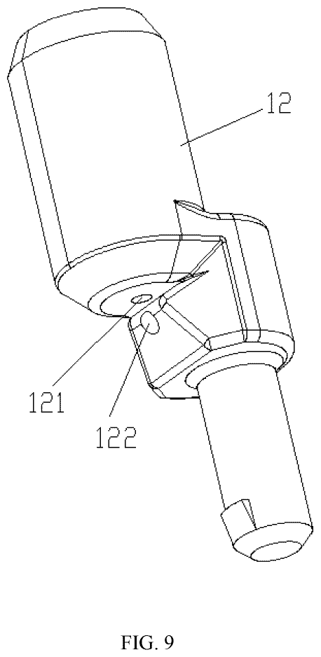

FIGS. 1-9 provide an aroma diffuser according to an embodiment of the present disclosure, which includes an atomizing assembly 1 and a barrel body 2 that are connected to each other. The atomizing assembly 1 includes an end cover 10 and a base 11 connected to the end cover 10, the barrel body 2 includes an outer barrel 20 and an inner barrel 21 provided in the outer barrel 20, a positioning member is provided between an inner wall of the outer barrel and an outer wall of the inner barrel, the positioning member includes a positioning groove formed in the outer wall of the inner barrel, and a positioning block provided on the inner wall of the outer barrel, the positioning block is in sliding fitting with the positioning groove, a clamping member is also provided between the inner wall of the outer barrel and the outer wall of the inner barrel; the positioning member includes a clamping groove provided in the inner wall of the outer barrel, and a clamping lug provided on the outer wall of the inner barrel, and the clamping lug is in snap fitting with the clamping groove. As shown in FIG. 3 and FIG. 5, these structures enable the inner barrel 21 and the outer barrel 20 to be located opposite to each other and fit closely, and the inner barrel 21 may not rotate or fall out relative to the outer barrel 20. A hanging structure which is over-turned outward is provided at a rim of the inner barrel 21, the hanging structure is configured for hanging at a rim of the outer barrel 20, and an annular recess is formed at the rim of the outer barrel 20 and matches with the hanging structure. In this embodiment, the hanging structure, the positioning groove and the clamping lug are integrally formed with the inner barrel 21, and the single integrated structure of the inner barrel 21 can achieve the functions such as hanging, positioning and clamping at the same time. An oil storage bottle 22 connected to the base 11 is also provided in the inner barrel 21. A connection mode between the base 11 and the oil storage bottle 22 may include, but is not limited to, a threaded connection mode. The oil storage bottle 22 is mainly configured for storing a liquid essential oil. An atomizing core 12 is provided in the base 11. The atomizing core 12 is located inside a mouth of the oil storage bottle 22 and is further provided with an air outlet 121 and a liquid outlet 122. The air outlet 121 and the liquid outlet 122 correspond to each other to form an included angle therebetween. An oil suction pipe 13 connected to the atomizing core 12 is provided at one end of the atomizing core 12. The oil suction pipe 13 is communicated with the liquid outlet 122, and one end of the oil suction pipe 13 extends to the bottom of the oil storage bottle 22, enabling the liquid essential oil to be sucked into the atomizing core 12 and then flows out through the liquid outlet 122. When the oil storage bottle 22 is located in the inner barrel 21, the oil storage bottle 22 is placed inside the inner barrel 21 by being connected to the base 11. The outer wall of the end cover 10 is in threaded connection with the inner wall of the outer barrel 20, the inner wall of the end cover 10 is in threaded connection with the outer wall of the base 11, and to facilitate unscrewing, vertical stripes are provided on the outer surface of the end cover as the friction surfaces. In addition, a limiting raised ring 101 is also provided at the lower end of the end cover 10. The limiting raised ring 101 is limited between the inner barrel 21 and the base 11, and thus, an airflow channel 4 for an airflow to pass through is formed between the inner wall of the inner barrel 21 and the outer wall of the oil storage bottle 22. An air inlet pipe 231 communicated with the airflow channel 4 is also provided at the bottom of the inner barrel 21, and an air outlet end of the air inlet pipe 231 is higher than a bottom surface of the inner barrel 21, preventing part of refluxed liquid from flowing into the air inlet pipe 231. Further, a baffle is further provided above the air outlet end, and the air inlet pipe 231 and the baffle are integrally formed with the inner barrel 21. An air inlet hole 110 communicated with the airflow channel 4 is formed in one end inside the base 11, the other end of the atomizing core 12 is communicated with the other end of the air inlet hole 110, and the air outlet 121 is communicated with the air outlet hole 110. Since the limiting raised ring 101 extends into the airflow channel 4 in a limiting manner, to avoid the limiting raised ring 101 from blocking the air from being introduced through the air inlet hole 110, a horizontal plane at the lower end of the limiting raised ring 101 is higher than the upper end of the air inlet hole 110. An air pump 23 for providing an airflow is also provided inside the outer barrel 20. The air pump 23 is mainly configured for pumping a high-speed airflow into the atomizing core 12. This high-speed airflow will be ejected from the air outlet 121 of the atomizing core 12, and thus, the liquid essential oil flowing out of the liquid outlet 122 is atomized. The inner barrel 21 is located above the air pump 23, and an air conveying pipe 230 connected to the air pump 23 is provided at one end of the air pump 23, while the other end of the air conveying pipe 230 is communicated with the air inlet pipe 231 on the inner barrel 21. When the liquid essential oil needs to be atomized, the air pump 23 starts to pump the high-speed airflow into the airflow channel 4 of the inner barrel 21 through the air conveying pipe 230 via the air inlet pipe 231. The high-speed airflow enters the atomizing core 12 from the air inlet hole 110 through the airflow channel 4 and is then ejected from the air outlet 121, forming a negative pressure at the liquid outlet 122, thereby enabling the liquid essential oil sucked up by the oil suction pipe 13 to flow out of the liquid outlet 122 of the atomizing core 12. The liquid essential oil is accelerated to collide by the high-speed airflow and rapidly atomized into essence oil molecule particles, and meanwhile, the liquid essential oil in the oil storage bottle 22 is continuously sucked to the liquid outlet 122 due to a negative pressure effect, and thus, the liquid essential oil in the oil storage bottle 22 is continuously atomized.

A partition plate 112 is also provided in the base 1 and integrally formed with the base 11. In order to enable the atomized essential oil molecular particles to diffuse from the bottle into the air, a plurality of diffusion holes 113 penetrating through the oil storage bottle 22, is formed in the partition plate 112, and atomized essential oil particles are pushed by the airflow and then blown upward from the diffusion holes 113. A gasket 114 which is configured for ensuring the airtightness between the base 11 and the end cover 10 is also provided in the base 11, and the gasket 114 is provided on a side wall of the base 11. Because the limiting raised ring 101 extends into the airflow channel 4 in a limiting manner, after the end cover 10 is connected to the base 11, the limiting raised ring 101 is fitted with the side wall of the base 11, thereby positioning the gasket 114 and avoiding the gasket 114 from falling off. To prevent the liquid essential oil particles that have not been completely atomized from blowing out from the diffusion holes 113 and then blowing out from the end cover 10 due to the push of the airflow, a fog baffle frame 14 is provided on the partition plate 112. The fog baffle frame 14 includes a fog baffle plate 140 and a plurality of fog outlets 141. A limiting column 142 is provided at the bottom of the fog baffle plate 140. The partition plate 112 is provided with a limiting hole 115 corresponding to the limiting column 142. The limiting column 142 is in limited fitting with the limiting hole 115. The fog baffle plate 140 is umbrella-shaped and located above the diffusion holes 113. The fog outlets 141 are evenly distributed around the fog baffle plate 140. A fog outlet channel 16 is formed between the inside of the end cover 10 and the fog baffle frame 14. The fog outlet holes 17 communicated with the fog outlet channel 16 are further formed in the upper end of the end cover 10 and configured for diffusing the atomized essential oil into the air. The whole fog baffle frame 14 is installed on the partition plate 112 by making the limiting column 142 be in limited fitting with the limiting hole 115. In addition, the fog baffle plate 140 is umbrella-shaped and is just located above the diffusion holes 113, while the fog outlets 141 are evenly distributed around the fog baffle plate 140. Therefore, after the atomizing core 12 blows out the atomized essential oil particle molecules, even large liquid essential oil molecules that have not been fully atomized will be blown out along with them through the diffusion holes 113. Since the umbrella-shaped fog baffle plate 140 is provided above the diffusion holes 113, the large liquid essential oil molecules that have not been atomized will also be blocked by the fog baffle plate 140 and continue to flow back into the oil storage bottle 22 through the diffusion holes 113. Meanwhile, the granular essential oil molecules that have been atomized will still be blown out from the fog outlets 141 on the side edge of the fog baffle plate 140 even if they are blocked by the fog baffle plate 140. A flog outlet channel 16 is formed between the inside of the end cover 10 and the fog baffle frame 14, and the upper end of the end cover 10 is also provided with the fog outlet holes 17 that is communicated with the fog outlet channel 16 and configured for diffusing the atomized essential oil into the air. Therefore, the granular essential oil molecules blown out from the fog outlets 141 are diffused into the air through the fog outlet channel 16 and then through the fog outlet holes 17, thereby achieving the effect of aroma diffusion.

Because the airflow channel 4 through which the high-speed airflow passes is provided between the inner wall of the inner barrel 21 and an outer wall of the oil storage bottle 22, and an air inlet source of the air inlet hole 110 all comes from the high-speed airflow flowing through the airflow channel 4, to ensure the air intake efficiency of the air inlet hole 110, it is necessary to ensure the airtightness between the atomizing assembly 1 and the inner barrel 21, thereby ensuring the airtight effect of the airflow channel 4. Therefore, the limiting device is provided between the atomizing assembly 1 and the barrel body 2, wherein the limiting device is provided with at least one sealing member 3 that is in limited fitting with the limiting device. Therefore, when the atomizing assembly 1 is connected to the barrel body 2, the sealing member 3 can be configured for horizontally sealing the gap between the barrel body 2 and the atomizing assembly 1, thereby ensuring the airtightness between the atomizing assembly 1 and the inner barrel 21 and preventing the leakage of the airflow in the airflow channel 4.

In this embodiment, the limiting device is located at the top of the inner barrel 21. The limiting device is an annular limiting groove 210 located at the top of the inner barrel 21. The sealing member 3 that is installed in limited fitting with the annular limiting groove 210 is provided inside the annular limiting groove 210. The sealing member 3 includes, but is not limited to, a sealing ring, a sealing loop or other sealing means, and the sealing member 3 has a cross section which includes, but is not limited to, a circular cross section, an oval cross section or a quadrilateral cross section. The sealing member 3 has a thickness greater than the depth of the annular limiting groove 210. When the atomizing assembly 1 is connected to the barrel body 2, the atomizing assembly 1 and the sealing member 3 are fitted vertically and sealed, which is equivalent to that the lower end surface of the atomizing assembly 1 is in horizontal fitting with the sealing member 3. Because the sealing member 3 is installed inside the annular limiting groove 210 located at the top of the inner barrel 21, it is equivalent to that the atomizing assembly 1 and the inner barrel 21 are sealedly connected through the sealing member 3. Therefore, the airtight effect of the airflow channel 4 is effectively ensured, and the atomization effect of the liquid essential oil in the atomizing core 12 is ensured by ensuring the air intake efficiency of the air inlet hole 110. When the atomizing assembly 1 is fitted with the sealing member 3, it is equivalent to that the lower end surface of the end cover 10 in the atomizing assembly 1 is in fitting with the sealing member 3. However, to further ensure the airtightness of the airflow channel 4, the end cover 10 can also be connected with the inner barrel 21 in a limiting manner by limiting the limiting raised ring 101 between the base 11 and the inner barrel 21, in other words, the inner barrel 21 and the airflow channel 4 can be sealed by the limiting raised ring 101, so as to ensure direct air intake of the airflow in the airflow channel 4 through the air inlet hole 110 in the base 11. However, the sealing member 3 is horizontally provided between the top end of the inner barrel 21 and the end cover 10. Therefore, to effectively ensure the sealing effect between the inner barrel 21 and the atomizing assembly 1 again after the sealing member 3 is installed, the end cover 10 in the atomizing assembly 1 is also provided with an annular table 102 corresponding to the annular limiting groove 210. After the atomizing assembly 1 is connected to the inner barrel 21, it is equivalent to that the end surface of the annular table 102 in the end cover 10 is in horizontal fitting with the sealing member 3. In addition, when the end cover 10 is properly connected with the outer barrel 20 and the inner barrel 21 in a limiting manner, because the thickness of the sealing member 3 is greater than the depth of the annular limiting groove 210, the annular table 102 located on the end cover 10 will press the sealing member 3 to a certain extent from top to bottom, so that the tight connection between the sealing member 3 and the annular table 102 is fully ensured, thereby fully ensuring the connection tightness between the inner barrel 21 and the end cover 10. Therefore, the gap between the inner barrel 21 and the atomizing assembly 1 can be horizontally sealed by the sealing member 3, effectively preventing the leakage of the airflow in the airflow channel 4 inside the inner barrel 21, enabling the high-speed airflow in the airflow channel 4 to all enter the atomizing core 12 through the air inlet hole 110, thereby effectively improving the atomization efficiency of the atomizing core 12 in absorbing the liquid essential oil from the oil suction pipe 13, and also greatly improving the aroma diffusion effect of the whole aroma diffuser. It should be understood that, in other embodiments, this limiting device may also be located at the top of the outer barrel 20.

In addition, the annular limiting groove 210 is horizontally formed in the top end of the inner barrel 21, the sealing member 3 is also horizontally installed in the annular limiting groove 210 in a limiting manner, and the surface of the annular table 102 on the end cover 10 is also a flat surface. In this way, even when the annular table 102 presses the sealing member 3 from top to bottom, and even when the atomizing assembly 1 and the barrel body 2 are installed or disassembled in a rotating manner, the sealing member 3 will not be distorted and hence deformed along the rotation, and it is even less likely to cause the sealing member 3 to fall off from the annular limiting groove 210. Therefore, the sealing effect between the upper end surface of the inner barrel 21 and the lower end surface of the end cover 10 can be ensured by the sealing member 3 in both a horizontal plane and a vertical plane, the usage stability of the sealing member 3 can also be ensured, and the service life is prolonged.

To meet a power supply demand of the air pump 23, a battery assembly 5 is also provided between the inner barrel 21 inside the outer barrel 20 and the air pump 23. The battery assembly 5 is electrically connected to the air pump 23 for supplying power to the air pump 23. Alternatively, the built-in battery assembly 5 may be omitted if no power supply is needed according to an actual demand, or an external power supply may also be configured for supplying power to the air pump 23.

A base 6 fixedly connected to the barrel body 2 is provided at the lower end of the barrel body 2, and a control module for controlling the aroma diffuser is also provided in the base 6.

The present disclosure has the following working principle or beneficial effect: by providing the sealing member 3 at one end, connected to the atomizing assembly 1, of the barrel body 2, when the atomizing assembly 1 is connected to the barrel body 2, the atomizing assembly 1 and the sealing member 3 are fitted vertically and sealed, so that the airtightness between the barrel body 2 and the atomizing assembly 1 can be effectively ensured, and the the leakage of the airflow in the barrel body 2 can be effectively avoided, thereby effectively improving the atomization efficiency of the atomizing core 12 for the liquid essential oil and also improving the aroma diffusion effect of the whole aroma diffuser.

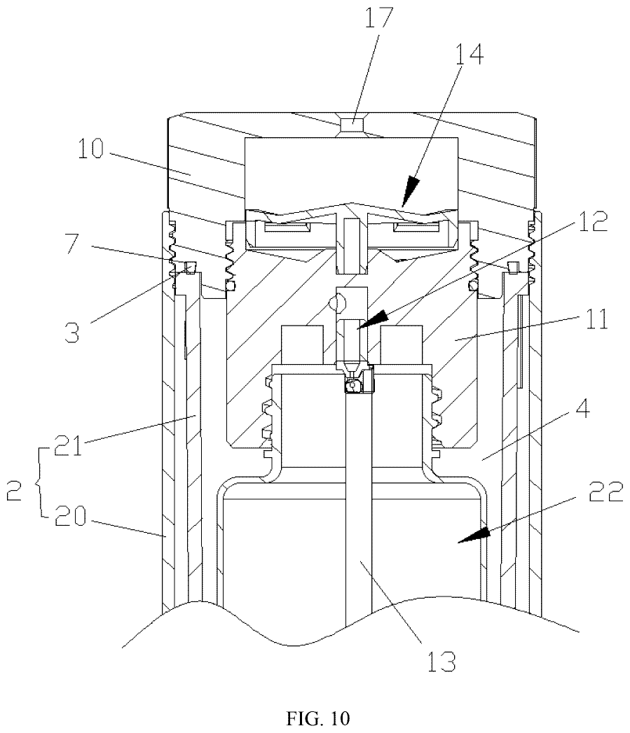

Referring to FIG. 10, an aroma diffuser is provided according to an embodiment of the present disclosure, which includes an atomizing assembly 1 and a barrel body 2 that are connected to each other. The barrel body 2 includes an outer barrel 20 and an inner barrel 21 provided in the outer barrel 20, a limiting device is provided between the atomizing assembly 1 and the barrel body 2, and the limiting device is provided with at least one sealing member 3 that is in limited fitting with the limiting device. This embodiment is different from the embodiment shown in FIGS. 1-9 in that the limiting device in this embodiment is located at the lower end surface of the end cover 10 in the atomizing assembly 1. The limiting device is also an annular limiting groove 210, but the annular limiting groove 210 is located at the lower end of the end cover 10, wherein the sealing member 3 is equivalent to be installed on the lower surface of the end cover 10 by limited installation with the annular limiting groove 210. In this way, when the end cover 10 in the atomizing assembly 1 is connected to the barrel body 2, the sealing member 3 located at the lower end of the end cover 10 is fitted with the upper end surface of the inner barrel 21 in a horizontal direction, which is equivalent to that the sealing member 3 may be configured for horizontally sealing a gap between the barrel body 2 and the end cover 10, so that the atomizing assembly 1 and the inner barrel 21 are connected in a sealed manner, and the airtightness of the airflow channel 4 is also ensured. Because the sealing member 3 is installed at the lower end of the end cover 10, to prevent the sealing member 3 from falling off from the annular limiting groove 210 located at the lower end of the end cover 10 due to gravity, the annular limiting groove 210 in this embodiment is provided as a dovetail groove, so that the sealing member 3 can be effectively limited.

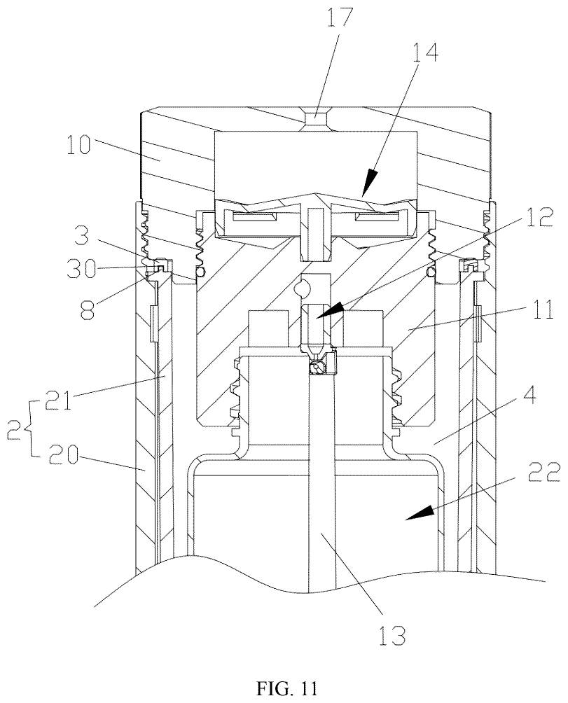

Referring to FIG. 11, an aroma diffuser is provided according to an embodiment of the present disclosure, which includes an atomizing assembly 1 and a barrel body 2 that are connected to each other. The barrel body 2 includes an outer barrel 20 and an inner barrel 21 provided in the outer barrel 20, a limiting device is provided between the atomizing assembly 1 and the barrel body 2, and the limiting device is provided with at least one sealing member 3 that is in limited fitting with the limiting device. This embodiment is different from the embodiment shown in FIGS. 1-9 in that the limiting device in this embodiment is an annular boss 8 located at the top of the inner barrel 21, and an annular groove 30 is provided in the bottom of the sealing member 3. Through the limited fitting between the annular groove 30 and the annular boss 8, the sealing member 3 may be installed on the annular boss 8 in a limiting manner, which is equivalent to that the sealing member 3 is installed on the top surface of the inner barrel 21. In this way, when the atomizing assembly 1 is in contact with the barrel body 2, the sealing member 3 can be configured for horizontally sealing a gap between the top surface of the inner barrel 21 and the atomizing assembly 1, so that the atomizing assembly 1 and the inner barrel 21 are connected in a sealed manner, the airtightness of the airflow channel 4 is also further ensured, and the atomization effect of the liquid essential oil in the atomizing core 12 is further ensured.

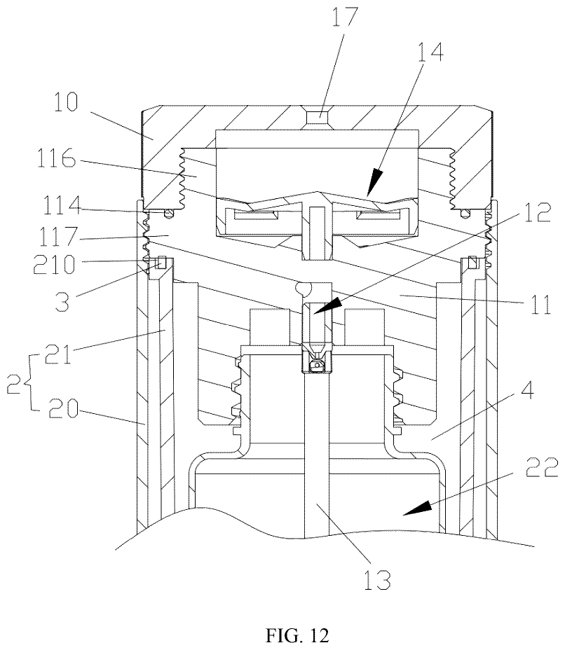

Referring to FIG. 12, an aroma diffuser is provided according to an embodiment of the present disclosure, which includes an atomizing assembly 1 and a barrel body 2 that are connected to each other. The atomizing assembly 1 includes an end cover 10 and a base 11 connected to the end cover 10, the barrel body 2 includes an outer barrel 20 and an inner barrel 21 provided in the outer barrel 20, an oil storage bottle 22 connected to the base 11 is also provided in the inner barrel 21, and the connection mode between the base 11 and the oil storage bottle 22 may include, but is not limited to a threaded connection mode. This embodiment is different from the embodiment shown in FIGS. 1-9 in that the base 11 in this embodiment is divided into an upper connecting portion 116 and a lower connecting portion 117, the upper connecting portion 116 has a diameter smaller than that of the lower connecting portion 117, an outer wall of the end cover 10 is in threaded connection with the outer wall of the upper connecting portion 116 of the base 11, an inner wall of the outer barrel 20 is in threaded connection with an outer wall of the lower connecting portion 117 of the base 11, and a gasket 114 is provided on the upper end surface of the lower connecting portion 117 and configured for being fitted with the lower end surface of the end cover 10, thereby ensuring the airtightness between the end cover 10 and the base 11. The sealing member 3 is also installed in the annular limiting groove 210 located at the top end of the inner barrel 21. Because the outer wall of the lower connecting portion 117 of the base 11 is in threaded connection with the inner wall of the outer barrel 20, the lower end surface of the lower connecting portion 116 of the base 11 is fitted with the upper surface of the sealing member 3, so that the airtightness between the top end of the inner barrel 21 and the base 11 is ensured, and the gap between the top surface of the inner barrel 21 and the atomizing assembly 1 can also be horizontally sealed by the sealing member 3.

An aroma diffuser according to an embodiment of the present disclosure includes an atomizing assembly 1 and a barrel body 2 that are connected to each other, the barrel body 2 includes an outer barrel 20 and an inner barrel 21 provided in the outer barrel 20, a limiting device is provided between the atomizing assembly 1 and the barrel body 2, and the limiting device is provided with at least one sealing member 3 that is in limited fitting with the limiting device. The limiting device in this embodiment is provided on the top surface of the outer barrel 20, so that when the atomizing assembly 1 is connected to the barrel body 2, the sealing member 3 is configured for horizontally sealing a gap between the top surface of the outer barrel 20 and the atomizing assembly 1.

An aroma diffuser according to an embodiment of the present disclosure includes an atomizing assembly 1 and a barrel body 2 that are connected to each other, the barrel body 2 includes an outer barrel 20 and an inner barrel 21 provided in the outer barrel 20, a limiting device is provided between the atomizing assembly 1 and the barrel body 2, the limiting device is provided with at least one sealing member 3 that is in limited fitting with the limiting device, and the atomizing assembly 1 includes an end cover 10 and a base 11. The limiting device in this embodiment is provided on the base 11. In this way, when the atomizing assembly 1 is connected to the barrel body 2, the sealing member 3 is configured for horizontally sealing a gap between the barrel body 2 and the base 11.

An aroma diffuser according to an embodiment of the present disclosure includes an atomizing assembly 1 and a barrel body 2 that are connected to each other, and the barrel body 2 includes an outer barrel 20 and an inner barrel 21 provided in the outer barrel 20. This embodiment is different from the embodiment shown in FIGS. 1-9 in that the outer barrel 20 and the inner barrel 21 in this embodiment are integrally formed, and thus being suitable for production where both the outer barrel 20 and the inner barrel 21 are made of plastic materials.

It should be eventually noted that the above embodiments are merely preferred embodiments of the present disclosure, but are not intended to limit the present disclosure. Although the present disclosure is described in detail with reference to the above embodiments, a person skilled in the art should understand that the technical solutions described in the foregoing embodiments may also be modified, or some of the technical features may be equivalently replaced. Thus, any modification, equivalent replacement, improvement and so on made within the spirit and principle of the present disclosure shall be encompassed by the protection scope of the present disclosure.

What is claimed is:

1. An aroma diffuser, comprising an atomizing assembly and a barrel body that are connected to each other, the barrel body comprising an outer barrel and an inner barrel provided in the outer barrel, and an airflow channel for an airflow to pass through being provided in the inner barrel, wherein a limiting device is arranged between the atomizing assembly and the barrel body, at least one sealing member is provided on the limiting device, and the sealing member is configured for horizontally sealing a gap between the barrel body and the atomizing assembly when the atomizing assembly is connected to the barrel body.

2. The aroma diffuser according to claim 1, wherein the limiting device is provided on a top surface of the outer barrel, and the sealing member is configured for horizontally sealing a gap between the top surface of the outer barrel and the atomizing assembly.

3. The aroma diffuser according to claim 1, wherein the limiting device is provided on a top surface of the inner barrel, and the sealing member is configured for horizontally sealing a gap between the top surface of the inner barrel and the atomizing assembly.

4. The aroma diffuser according to claim 1, wherein the atomizing assembly comprises an end cover and a base, an atomizing core is provided in the base, the airflow channel is communicated with the atomizing core, the limiting device is provided on the end cover, and the sealing member is configured for horizontally sealing a gap between the barrel body and the end cover.

5. The aroma diffuser according to claim 1, wherein the atomizing assembly comprises an end cover and a base, an atomizing core is provided in the base; the airflow channel is communicated with the atomizing core, the limiting device is provided on the base, and the sealing member is configured for horizontally sealing a gap between the barrel body and the base.

6. The aroma diffuser according to claim 1, wherein the limiting device is an annular groove, and the sealing member is provided in the annular groove and has a height greater than the depth of the annular groove.

7. The aroma diffuser according to claim 1, wherein the limiting device is an annular boss, and a groove that matches with the annular boss is formed in the bottom of the sealing member.

8. The aroma diffuser according to claim 1, wherein the atomizing assembly comprises an end cover and a base, a limiting raised ring is provided at a lower end of the end cover, and the limiting raised ring is limited between the inner barrel and the base when the atomizing assembly is connected to the barrel body.

9. The aroma diffuser according to claim 1, wherein a positioning member is provided between an inner wall of the outer barrel and an outer wall of the inner barrel.

10. The aroma diffuser according to claim 9, wherein the positioning member comprises a positioning groove formed in the inner wall of the outer barrel/the outer wall of the inner barrel, and a positioning block provided on the outer wall of the inner barrel/the inner wall of the outer barrel, and the positioning block is in sliding fitting with the positioning groove.

11. The aroma diffuser according to claim 1, wherein a clamping member is provided between the inner wall of the outer barrel and the outer wall of the inner barrel.

12. The aroma diffuser according to claim 11, wherein the positioning member comprises a clamping groove formed in the inner wall of the outer barrel/the outer wall of the inner barrel, and a clamping lug provided on the outer wall of the inner barrel/the inner wall of the outer barrel, and the clamping lug is in snap fitting with the clamping groove.

13. The aroma diffuser according to claim 1, wherein a hanging structure which is over-turned outwards is provided at a rim of the inner barrel, and the hanging structure is configured for hanging at a rim of the outer barrel.

14. The aroma diffuser according to claim 13, wherein a plurality of positioning grooves and a plurality of clamping lugs are provided on the outer wall of the inner barrel, and the hanging structure, the positioning grooves and the clamping lugs are integrally formed with the inner barrel.

15. The aroma diffuser according to claim 1, wherein an air inlet pipe communicated with the airflow channel is further provided at the bottom of the inner barrel, and an air outlet end of the air inlet pipe is higher than a bottom surface of the inner barrel.

16. The aroma diffuser according to claim 15, wherein a baffle is provided above the air outlet end.

17. The aroma diffuser according to claim 16, wherein the air inlet pipe and the baffle are integrally formed with the inner barrel.

18. The aroma diffuser according to claim 1, wherein friction surfaces are distributed on the outer surface of the atomizing assembly.
